(19) United States
(12) Patent Application Publication
(10) Pub. No.: US 2020/0129110 A1
(43) Pub. Date: Apr. 30, 2020

(54) FUNCTIONAL ANALYSIS OF HUMAN BRAIN USING FUNCTIONAL MAGNETIC RESONANCE IMAGING (FMRI) FOR ACUTE STRESS AND POST TRAUMATIC STRESS DISORDER (PTSD) MONITORING NEUROPLASTICITY

(71) Applicant: Translational Research Institute Pty Ltd as trustee for Translational Research Institute Trust, Woolloongabba (AU)

(72) Inventors: Carolyn Mountford, Robina (AU); Marcus Gray, Woolloongabba (AU)

(73) Assignee: Translational Research Institute Pty Ltd as trustee for Translational Research Institute Trust, Woolloongabba (AU)

(21) Appl. No.: 16/662,579

(22) Filed: Oct. 24, 2019

Related U.S. Application Data
(60) Provisional application No. 62/749,990, filed on Oct. 24, 2018.

(51) Int. Cl. A61B 5/00; A61B 5/026

(52) U.S. Cl. CPC A61B 5/4064; G06T 7/0012; A61B 5/026; A61B 5/7267

(57) ABSTRACT

A method and system for enabling a practitioner to detect acute stress disorder and PTSD using fMRI comprises looking at functional connectivity dynamics within the visual pathways, limbic, thalamic, paralimbic and prefrontal brain networks observed when individuals are viewing visual images of different facial expressions.

### CROSS-REFERENCE TO RELATED APPLICATIONS

This application claims priority to U.S. Ser. No. 62/749,990 filed Oct. 24, 2018 which is incorporated by reference herein.

### BACKGROUND OF THE INVENTION

The present invention relates to use of functional Magnetic Resonance Imaging (fMRI) to detect and monitor altered brain functional dynamics underlying acute stress and post-traumatic stress disorder (PTSD).

PTSD is a serious disorder affecting many combat soldiers, as well as others exposed to extremely stressful conditions, even in training conditions short of actual combat situations. Special operations soldiers are particularly at risk, during training and combat, due to the extremely stressful training conditions and combat conditions. Additionally, many other persons who have not been exposed to combat or extreme training situations, but who may otherwise have experienced extreme stress may also develop PTSD, particularly if the exposure to stressful conditions continues. Simply put, PTSD is a behavioral manifestation of underlying changes in brain function. These changes are likely evident well before PTSD is diagnosed, and are linked to severe, and/or prolonged stress exposure. Children in their early formative years may be particularly vulnerable to stress-associated change, such that highly traumatic events are likely to severely impact their emotional mental health, and increase the risk for PTSD later in life. The ability to detect changes in brain function reflecting severe and/or prolonged stress exposure may be very valuable, as it can allow intervening action, such as preventing additional stress exposure, and if necessary initiating behavioral or pharmacological therapy.

### SUMMARY OF THE INVENTION

In accordance with the present invention, a method and system are provided for using fMRI to detect altered brain function underlying following severe and/or prolonged stress exposure. These changes underlie PTSD, and may also be present in apparently healthy people who are highly anxious, or who have experienced severe or prolonged stress exposure. In these people, altered brain functional dynamics may represent stress-associated change which increases the risk of developing PTSD in the future.

An important realization in our approach is that we can measure the brain functional dynamics highlighted above. Specifically, processing visual images with emotional content will engage the circuits of relevance. Their function can then be examined for signs of stress-associated change. For example, it's recognized that viewing images of fearful or anxious facial expressions activates visual, limbic and prefrontal brain regions. These responses reflect emotional content (e.g. responses to fear expressions differ from happy expressions), and can be used to differentiate between people. We have discovered two important findings. Firstly, examining fear responses (versus happy) engages the posterior cingulate gyms (PCG) significantly more in special ops soldiers than in healthy controls. Secondly, these PCG responses in special ops soldiers are similar to responses in PTSD sufferers. Thus, changes in PCG response to visual fear imagery may represent early stages of a multi-step process which occurs within PTSD, especially if the exposure is extremely stressful, repeated, and absent treatment and therapy.

Early detection of the effect of acute stress producing events on individuals is important and valuable. The sooner medical practitioners are able to detect changes in brain function during an acute stress event, the sooner intervention can occur to protect that person from acute stress and neurobiological changes that precede PTSD. It is much easier to treat a vulnerable person exposed to acute stress in the early stages, before the person advances to PTSD, than it is to treat a person who has PTSD. In some cases of PTSD, a person may never recover and any recovery is only partial because the person cannot rid themselves of the trauma of the event. Accordingly, early detection and in some cases therapy is extremely important in managing mental health in people exposed to acute stress causing events.

For example, in military training of special ops soldiers, the soldiers are exposed to extreme acute stress provoking events. All healthy persons are negatively impacted by severe stress and trauma, however some soldiers experience these events without going on to developing PTSD. Other are significantly affected, and it may be advisable to limit severe stress exposure in these people. Military personnel would like to be able to monitor special ops trainees in order to ensure that vulnerable soldiers are treated with appropriate care and respect, for the benefit of the individuals, the military, and society.

It is known that for some, the mere passage of time is sufficient to cure the person of the effects of a traumatic event. Other people need therapy such as counseling or medication. Using the method and system of the invention would provide a great benefit to society and to the individuals whose early detection would result in intervention and treatment.

### BRIEF DESCRIPTION OF THE DRAWINGS

FIG. 1 shows on the left the preprocessing of functional Magnetic Resonance Imaging (fMRI) scans of the brain typically applied during statistical parametric mapping (SPM) of brain function. A sessions data is realigned, spatially normalized and smoothed before a design matrix is created under the general linear model. A general linear model is constructed for each person, identify the brains response to fearful, happy and neutral expressions (i.e. parameter estimates). These parameters estimates are taken as the input data (features) a classification model uses to differentiate between adults with PTSD from adults without PTSD (healthy adults).

FIG. 2 section 1 shows two different emotion-related stimuli (facial expression images); a happy and a fearful/stressful expression. Parameter estimates (see FIG. 1) show the pattern of brain response to each type of facial expression (FIG. 2 section 2) and the difference in responses to happy versus fearful expressions. These parameters reflect how different the brain response to happy versus fearful stimuli is, at every brain location (larger parameters reflecting larger differences in brain response).

FIG. 2 section 2 illustrates a thresholded parameter map with only the largest differences represented by orange clusters. These parameter maps were then parcelated into regional clusters throughout the brain (FIG. 2 section 3). Each region is represented by the mean value of all parameter values within that region. This reduces (summarizes) the pattern of brain responses. These averaged estimates of different regional brain responses were then used as “features” (data for classification). This represented an initial, minimally informed method for feature selection, and utilized a standard anatomical template (Mageleburg 758 Atlas). A supervised machine learning classifier was then developed (section 4) to differentiate groups of participants based on their brain differential responses to fearful and happy stimuli. We used the “Classification Learner” toolbox in Matlab to train a range of classifiers under differing statistical approaches (e.g. discriminant function classifiers, support vector machine classifiers, k nearest neighbor classifiers etc.) A classification accuracy of 87.5% was obtained for support vector machine (linear) and discriminant function (linear) classifiers when differentiating PTSD sufferers from healthy controls. The average accuracy over our 23 classifiers was 77.1%±4.7%. The experiment involved 86 participants (21 with PTSD and 65 with non-PTSD).

FIG. 3 is a sketch of the human brain illustrating the current understanding of how visual information is processed by the brain. Visual information from the eye is projected to the primary visual cortex (area V1) at the back of the brain. Here is it split into a dorsal visual pathway (DVP) and a ventral visual pathway (VVP). We examined activity within both the DVP and VVP relevant during the processing of visual information, and within the limbic/paralimbic system (LPS) such as the amygdala (Amy) and prefrontal cortex (PFC) regions which modulate visual processing with emotive content. By examining correlated brain function within these regions, we examined how the brain passed information between these areas when processing visual images. This approach uses knowledge of brain systems and function gained over the past century to more intelligently select features.

FIG. 4 shows a method for identifying functional connectivity within the brain called a psychophysiological interaction (PPI) which allows us to see how changes in our experimental factor (fearful faces versus happy faces) alters the correlation between a specific chosen brain region and all other regions within the brain. This modeling is conducted after the original general linear modeling of brain responses, and involves creating one or more new statistical models for each participant. This allows the inter-regional connectivity within the ventral visual pathway (VVP), limbic/paralimbic system (LPS) and prefrontal cortex (PFC) to be examined.

FIG. 5 shows the introduction of additional noise regressors. On the left is a design matrix containing 3 task regressors (fearful (or stress inducing), happy and neutral expressions (non-stress inducing)) and 27 noise regressors taken from areas which did not contain brain tissue, but instead included cerebrospinal fluid. Signal changes within these areas therefore reflect noise, not neural activity, allowing better estimation and separation of noise and brain function in the rest of the brain. Noise regressors were created from the superior ventricles, caudal ventricles, inferior ventricles and the quadrigeminal cistern, and are shown in FIG. 5. Modeling noise regressors significantly improved the model fit.

FIG. 6 shows fMRI images for a single participant, and on the right a brain atlas which illustrates areas where functional connectivity to the primary visual area V1 (red circle in upper left fMRI image) was significantly changed by fearful image stimulation. This is an example of a PPI model for a particular person, and for a single brain region (area V1 in the visual processing stream). This model produces a parameter for every spatial brain location within the brain (many thousand parameters), each showing how the visual stimuli changed the correlations between that area and area V1. We then extended this modeling beyond brain area V1. We constructed 32 different PPI models for each person scanned, based on 32 seed locations from the ventral and dorsal visual streams, limbic, paralimbic and prefrontal regions.

FIG. 7 shows the location of the majority of the brain locations just referred to. This represents a much more informed method for feature selection than the parcellation approach illustrated in FIG. 2, and draws on knowledge cumulatively gained by many thousands of neuroscientists over many hundreds of years.

FIG. 8 illustrates our approach for informed fMRI classifiers. We use the parameters from a range of PPI models which identify how brain networks process visual images with affective content. These parameters are sensitive to how information flows through the visual processing pathways, and to how limbic and prefrontal regions modify these information flows. Machine learning classifiers given these features can then learn to differentiate participant groups, for example people with PTSD from those without. Finally, once classification models are obtained, they can then be used with new individuals, to give an indication if changes in visual processing typical of PTSD or acute stress are evident.

FIG. 9 is a block diagram of an MRI system which can be used to obtain the data to practice the invention.

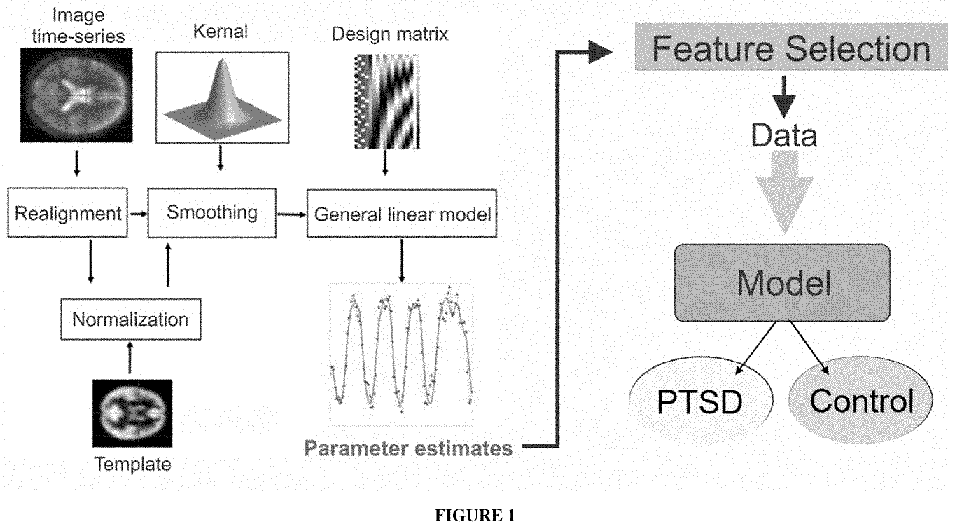

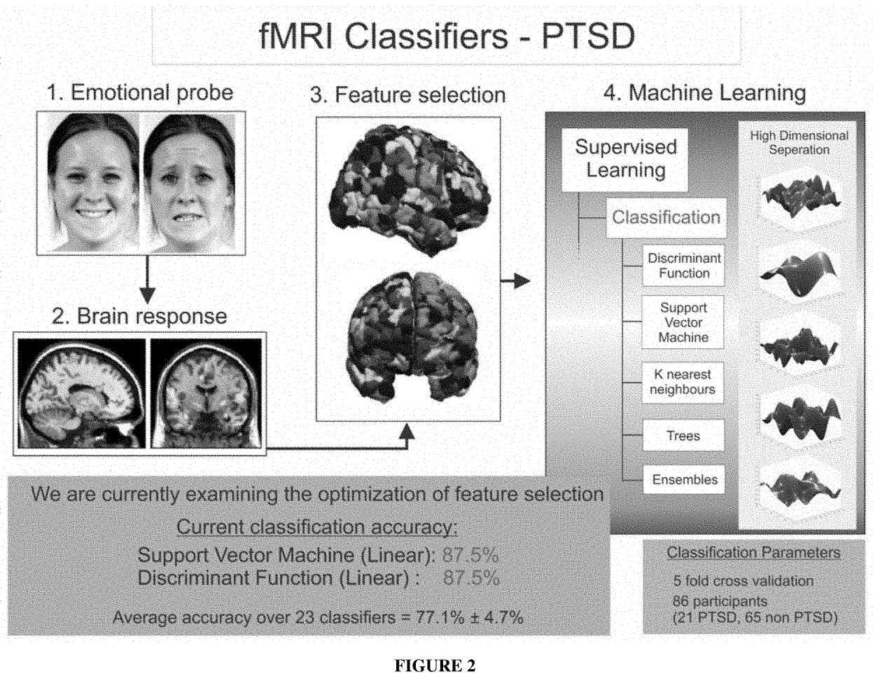

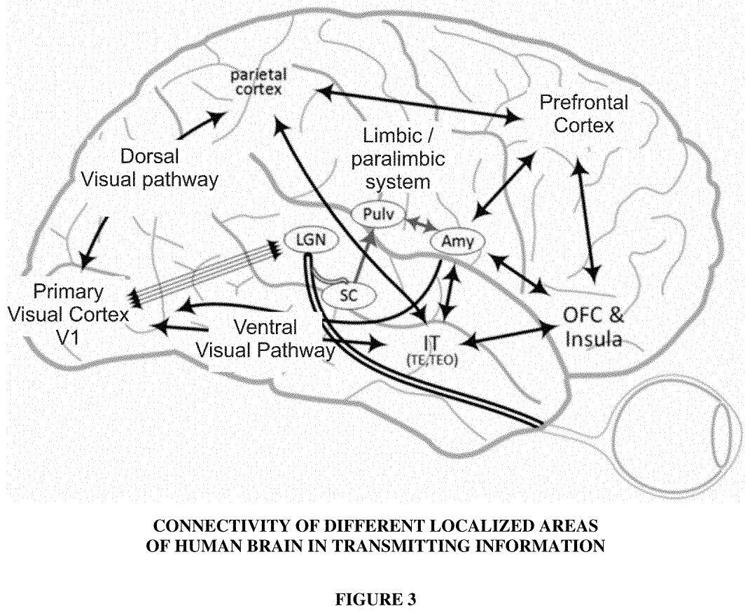

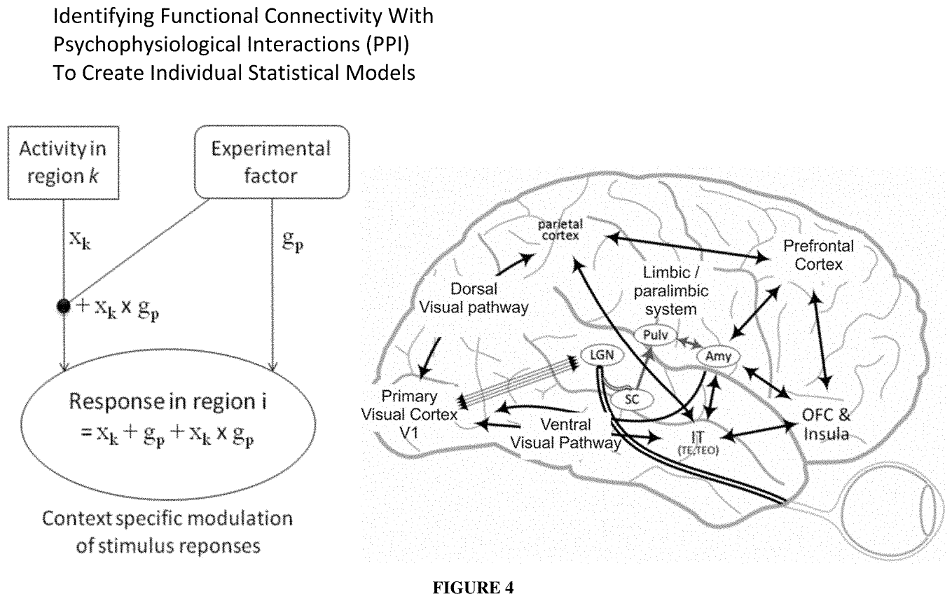

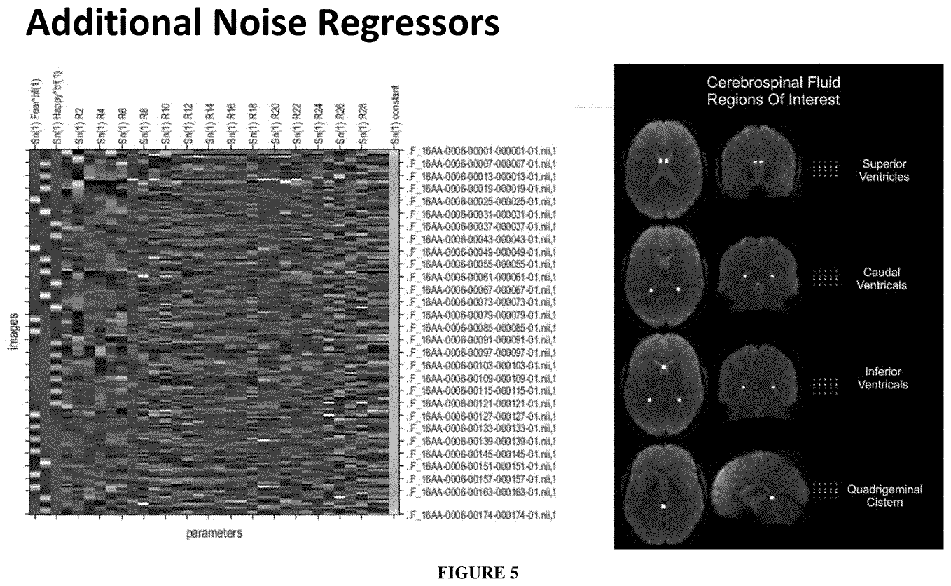

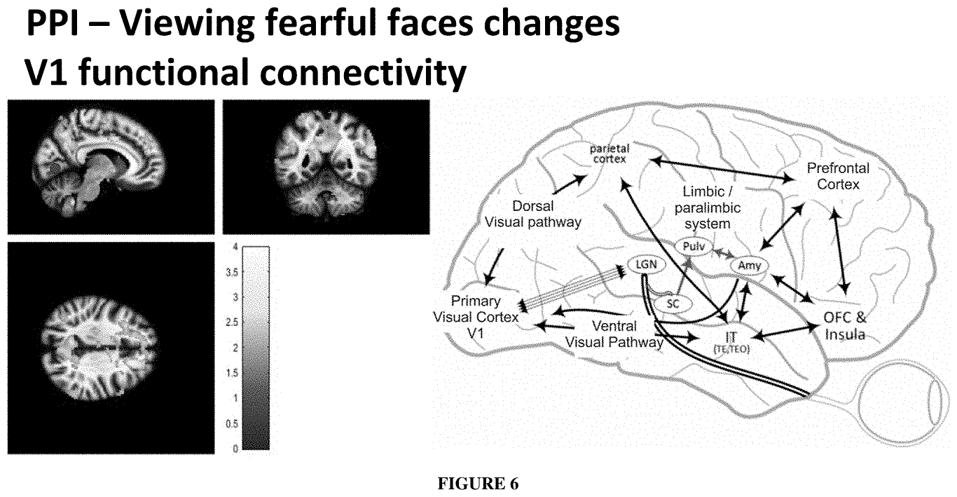

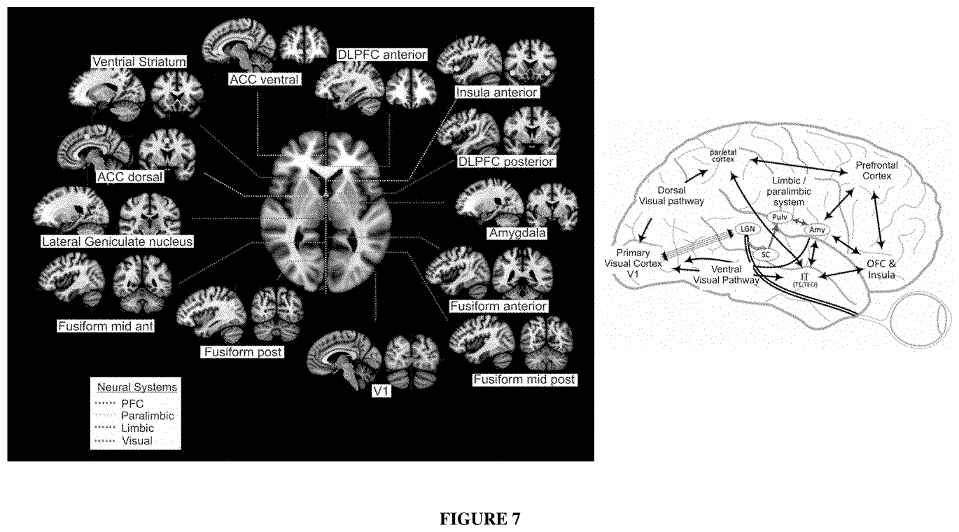

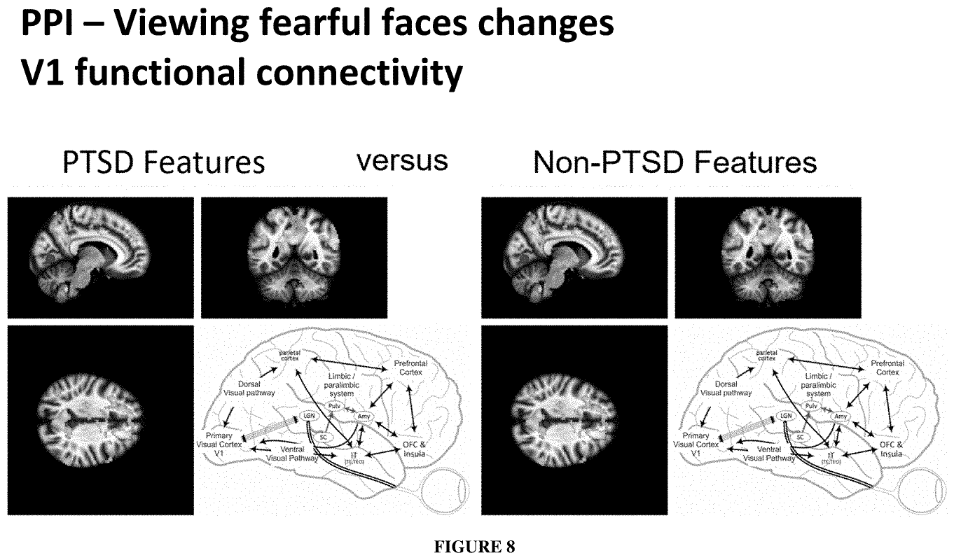

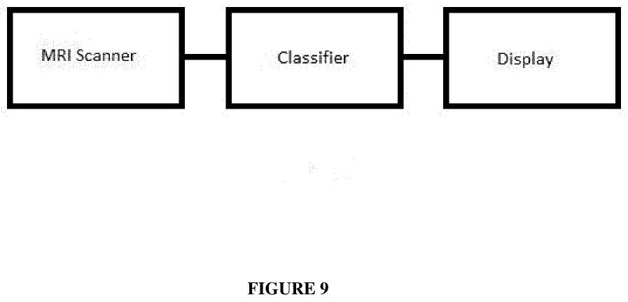

### BRIEF DESCRIPTION OF THE PREFERRED EMBODIMENT

A preferred embodiment of the invention will be described by way of example only, but the invention is not limited to this embodiment.

The invention provides a method of monitoring how a person with PTSD responds to emotional stimuli to create a classifier to distinguish healthy controls from PTSD (emotion processing for PTSD) based on fMRI functional brain dynamics, comprising: using an MRI scanner and an fMRI sequence to functionally image whole brain function within individuals who are viewing images of different classes of facial expressions; modeling individual brain responses to each class of facial expression via general linear modeling in an event related design; calculating dynamic functional connectivity changes within a network of brain regions as a function of different classes of facial expressions images; generating as characteristic features the functional connectivity dynamics induced by viewing each class of facial expression; and using a machine learning classifier to create an emotion processing for PTSD classifier tool usable to compare and differentiate individuals based on the characteristic features reflecting the functional connectivity dynamic changes to enable a practitioner to differentiate and identify individuals with PTSD, and individuals who have undergone neurological change following severe or prolonged stress exposure from otherwise healthy individuals by reference to the characteristic features.

The method the brain regions may include at least one of the ventral and dorsal visual pathways (VVP & DVP), limbic/paralimbic system (LPS), lateral genituclate nucleus (LGN) and prefrontal cortex (PFC). The functional connectivity may comprise functional connectivity within at least one of the visual pathways, thalamic, limbic/paralimbic and prefrontal brain networks. The functional connectivity based classifiers may be sensitive to stress associated change within: vulnerable but otherwise healthy individuals who following extreme or prolonged stress exposure; highly anxious individuals; and individuals with PTSD. The different classes of facial expressions may include stress-inducing images and non-stress-inducing images.

The invention provides a system for creating a PTSD emotion processing classifier for enabling the detection of stress-induced effects in functional brain dynamics within individuals using fMRI measurements during viewing of images of different facial expressions, the system comprising: an MRI scanner and an fMRI sequence to functionally image whole brain function within the individuals during a facial expression viewing task of different classes of visual images; a processor to (i) create a general linear model of individual brain responses to each class of visual image; (ii) calculate functional connectivity dynamic changes within a network of brain regions as a function of different classes of visual images; and (iii) generate characteristic features reflecting functional connectivity dynamics induced by viewing of different facial expressions; and a machine learning classifier to create a PTSD emotion processing classifier tool usable to compare and differentiate individuals based on characteristic features reflecting functional connectivity dynamic changes, to enable a practitioner to differentiate and identify individuals with PTSD, and individuals who have undergone neurological change following severe or prolonged stress exposure from otherwise healthy individuals by reference to characteristic features.

For information on how to create a general linear model, see cited reference 3.

The brain regions may include at least one of the ventral and dorsal visual pathways (VVP & DVP), limbic/paralimbic systems (LPS), lateral genituclate nucleus (LGN) and prefrontal cortex (PFC). The functional connectivity may comprise functional connectivity within at least one of the visual pathways, thalamic, limbic/paralimbic and prefrontal brain networks. The functional connectivity based classifiers may be sensitive to stress associated change within: vulnerable but otherwise healthy individuals who following extreme or prolonged stress exposure; highly anxious individuals; and individuals with PTSD. The different classes of facial expressions may include stress-inducing images and non-stress-inducing images.

The invention provides a method of enabling a practitioner to detect stress-induced effects to an individual from exposure to stress events, using fMRI, to detect whether an individual has PTSD, or acute stress disorder the method comprising: using an MRI scanner to obtain first fMRI images of selected regions of the brain of the individual while subjecting an individual to a potential stress causing event; and using a classifier to compare the first fMRI images obtained to second fMRI images obtained from individuals exposed to normal or happy events, to enable a practitioner to determine whether the first fMRI images detect functional connectivity between selected regions of the brain indicative of the individual having PTSD, or acute stress disorder.

The selected regions of the brain may include at least one of the ventral and dorsal visual pathways (VVP & DVP), limbic/paralimbic system (LPS), lateral genituclate nucleus (LGN) and prefrontal cortex (PFC). The functional connectivity may comprise functional connectivity within at least one of the visual pathways, thalamic, limbic/paralimbic and prefrontal brain networks. The magnitude of the change in functional connectivity may enable a practitioner to determine whether the individual has PTSD, or acute stress disorder.

The invention provides a system of enabling a practitioner to detect stress-induced effects to an individual from exposure to stress events, using fMRI, to detect whether an individual has PTSD, or acute stress disorder, the system comprising: an MRI scanner to obtain first fMRI images of selected regions of the brain of the individual while subjecting an individual to a potential stress causing event; and a classifier to compare the first fMRI images obtained to second fMRI images obtained from individuals exposed to normal or happy events, to enable a practitioner to determine whether the first fMRI images detect functional connectivity between selected regions of the brain indicative of the individual having PTSD, or acute stress disorder.

The selected regions of the brain may include at least one of the ventral and dorsal visual pathways (VVP & DVP), limbic/paralimbic system (LPS), lateral genituclate nucleus (LGN) and prefrontal cortex (PFC). The functional connectivity may comprise functional connectivity within at least one of the visual pathways, thalamic, limbic/paralimbic and prefrontal brain networks. The magnitude of the change in functional connectivity may enable a practitioner to determine whether the individual has PTSD, or acute stress disorder. The potential stress causing event may be an image of a person under stress.

fMRI Classifier Development—Materials and Methods

### Scanning Parameters.

BOLD weighted fMRI volumes were acquired on a Siemens MAGNETOM Prisma 3Tesla Magnetic Resonance Imaging scanner. The operating parameters of this echo-planar imaging sequence were: Volume TR 2.84s, TE 30 ms, 50 axial slices at 3 mm thickness, 2.95×2.95 mm in-plane resolution, flip angle 80 degrees, imaging frequency 123.26, Pixel bandwidth 2275, 87 phase encoding steps, field of View: 2080*2080 in-plane phase encoding direction: COL.

### Analytical Approach

Integral to this fMRI based classifier approach is that functional neural circuits relevant to disease associated change are experimentally challenged while brain activity is assessed. For example, in differentiating patients with PTSD, or high levels of acute stress from healthy adults, functional alterations in the processing of visual fear-associated images within the dorsal and ventral visual streams, limbic and prefrontal regions are explored and identified.

### Task Design and Acquisition.

We employed a visual task requiring participants to view pictures of human facial expressions, illustrating specific emotional expressions (fearful, happy or neutral). We used a block design, displaying 7 sequential images within a single expression category for approximately 1 second each (approx. 7 second blocks). 10 blocks of each expression category were displayed over approximately 8.3 mins. Prior to each scanning session, all participants were shown a scripted set of task instructions, and completed a short practice task with experimenter guidance and feedback to ensure accurate task performance. During brain scanning, stimuli were projected onto a screen behind the magnet bore from an MRI compatible projector and viewed via an angled mirror fitted to the MRI head coil.

fMRI Preprocessing

The fMRI data were pre-processed and analyzed using Statistical Parametric Mapping 12 (SPM12) software and MATLAB (version 8.2 2013b) (Friston et al., 2007). Raw images were realigned, screened for artifacts with Artrepair (version 4, Stanford University), and normalized via the segment routine (Ashburner 2007), prior to writing at 2 mm isotropic spatial resolution and smoothing with an 8 mm FWHM (full width at half maximum) kernel.

fMRI 1st Level Modelling

First level modeling (Friston et al., 2007), performed at the single subject level, included regressors for each expression (fearful, happy, neutral), 6 realignment parameters and 21 noise regressors taken from within the lateral ventricles and outside the brain. This allows the identification of neural regions engaged while viewing facial expressions.

### Modelling Psycho-Physiological Interactions (PPI's)

We then constructed PPI models (Friston et al, 1997) for each participant, based on the functional brain activity quantified in each 1st level model. We selected 32 seed regions within the brain within the dorsal visual pathways (DVP) and ventral visual pathway (VVP), limbic/paralimbic system (LPS) regions, lateral geniculate nucleus (LGN) and prefrontal cortex (PFC), based on models of visual-emotional processing (Silverstein & Ingvar, 2015). PPI interaction terms characterizing how the functional connectivity between each seed region and all other brain regions was changed by a function of task were calculated for each participant. From these models (32 per participant) we extracted parameters quantifying how the functional connectivity within the visual pathways, limbic, thalamic, paralimbic and prefrontal brain networks was altered when processing fear associated facial expressions.

fMRI Classifier Development

We then transferred our analysis to the Classifier Development Toolbox from Mathworks, in Matlab. We furnished our classifiers with features selected from PPI analysis of visual processing, and with 5-fold cross validation, trained families of classifiers. These included multiple classifier models within the following families: Decision tree classifiers, discriminant function classifiers, support vector machine classifiers, K-nearest neighbor's classifiers, logistic regression classifiers and ensemble classifiers. This approach produces fMRI based classifiers which are capable of differentiating PTSD patients from matched healthy controls with a classification accuracy of above 85%.

The same type of response was also observed for apparently healthy persons who were in a state of acute anxiety.

A preferred embodiment of a method and system for detecting PTSD and acute stress producing events using fMRI has been described, but the invention is not limited to this embodiment, and the invention is defined only by way of the following claims.

### REFERENCES CITED WHICH ARE INCORPORATED BY REFERENCE HEREIN

1. J. Ashburner, J. (2007). A fast diffeomorphic image registration algorithm. Neurolmage, 38(1):95-113.
2. Friston, K. J., Buchel, C., Fink, G. R., Morris, J., Rolls, E. and Dolan, R. (1997). Psychophysiological and modulatory interactions in neuroimaging. Neurolmage, 6:218-229.
3. Friston, K. J., Ashburner, J., Kiebel, S. J., Nichols, T. E. and Penny, W. D. (2007). Statistical Parametric Mapping: The Analysis of Functional Brain Images. Elsevier Academic Press.
4. Gray M A, Chao C Y, Staudacher H M, Kolosky N A, Talley N J, Holtmann G. (2018). Anti-TNFα therapy in IBD alters brain activity reflecting visceral sensory function and cognitive-affective biases. PLoS One, 13(3):e0193542.
5. Silverstein D N, Ingvar M. (2015). A multi-pathway hypothesis for human visual fear signaling. Front Syst Neurosci. 24; 9:101.

1. A method of monitoring how a person with PTSD responds to emotional stimuli to create a classifier to distinguish healthy controls from PTSD (emotion processing for PTSD) based on fMRI functional brain dynamics, comprising:
using an MRI scanner and an fMRI sequence to functionally image whole brain function within individuals who are viewing images of different classes of facial expressions;
modeling individual brain responses to each class of facial expression via general linear modeling in an event related design;
calculating dynamic functional connectivity changes within a network of brain regions as a function of different classes of facial expressions images;
generating as characteristic features the functional connectivity dynamics induced by viewing each class of facial expression; and
using a machine learning classifier to create an emotion processing for PTSD classifier tool usable to compare and differentiate individuals based on the characteristic features reflecting the functional connectivity dynamic changes to enable a practitioner to differentiate and identify individuals with PTSD, and individuals who have undergone neurological change following severe or prolonged stress exposure from otherwise healthy individuals by reference to the characteristic features.

2. The method of claim 1, wherein the brain regions include at least one of the ventral and dorsal visual pathways (VVP & DVP), limbic/paralimbic system (LPS), lateral genituclate nucleus (LGN) and prefrontal cortex (PFC).

3. The method of claim 1, wherein the functional connectivity comprises functional connectivity within at least one of the visual pathways, thalamic, limbic/paralimbic and prefrontal brain networks.

4. The method of claim 1, wherein functional connectivity based classifiers are sensitive to stress associated change within:
vulnerable but otherwise healthy individuals who following extreme or prolonged stress exposure;
highly anxious individuals; and
individuals with PTSD.

5. The method of claim 1, wherein the different classes of facial expressions include stress-inducing images and non-stress-inducing images.

6. A system for creating a PTSD emotion processing classifier for enabling the detection of stress-induced effects in functional brain dynamics within individuals using fMRI measurements during viewing of images of different facial expressions, the system comprising:
an MRI scanner and an fMRI sequence to functionally image whole brain function within the individuals during a facial expression viewing task of different classes of visual images;
a processor to (i) create a general linear model of individual brain responses to each class of visual image;
(ii) calculate functional connectivity dynamic changes within a network of brain regions as a function of different classes of visual images;
(iii) generate characteristic features reflecting functional connectivity dynamics induced by viewing of different facial expressions; and
a machine learning classifier to create a PTSD emotion processing classifier tool usable to compare and differentiate individuals based on characteristic features reflecting functional connectivity dynamic changes, to enable a practitioner to differentiate and identify individuals with PTSD, and individuals who have undergone neurological change following severe or prolonged stress exposure from otherwise healthy individuals by reference to characteristic features.

7. The system of claim 6, wherein the brain regions include at least one of the ventral and dorsal visual pathways (VVP & DVP), limbic/paralimbic systems (LPS), lateral genituclate nucleus (LGN) and prefrontal cortex (PFC).

8. The system of claim 6 wherein the functional connectivity comprises functional connectivity within at least one of the visual pathways, thalamic, limbic/paralimbic and prefrontal brain networks.

9. The system of claim 6, wherein functional connectivity based classifiers are sensitive to stress associated change within:
vulnerable but otherwise healthy individuals who following extreme or prolonged stress exposure;
highly anxious individuals; and
individuals with PTSD.

10. The system of claim 6, wherein the different classes of facial expressions include stress-inducing images and non-stress-inducing images.

11. A method of enabling a practitioner to detect stress-induced effects to an individual from exposure to stress events, using fMRI, to detect whether an individual has PTSD, or acute stress disorder the method comprising:
using an MRI scanner to obtain first fMRI images of selected regions of the brain of the individual while subjecting an individual to a potential stress causing event; and
using a classifier to compare the first fMRI images obtained to second fMRI images obtained from individuals exposed to normal or happy events, to enable a practitioner to determine whether the first fMRI images detect functional connectivity between selected regions of the brain indicative of the individual having PTSD, or acute stress disorder.

12. The method of claim 11, wherein the selected regions of the brain include at least one of the ventral and dorsal visual pathways (VVP & DVP), limbic/paralimbic system (LPS), lateral genituclate nucleus (LGN) and prefrontal cortex (PFC).

13. The method of claim 11 wherein the functional connectivity comprises functional connectivity within at least one of the visual pathways, thalamic, limbic/paralimbic and prefrontal brain networks.

14. The method of claim 11, wherein the magnitude of the change in functional connectivity enables a practitioner to determine whether the individual has PTSD, or acute stress disorder.

15. A system of enabling a practitioner to detect stress-induced effects to an individual from exposure to stress events, using fMRI, to detect whether an individual has PTSD, or acute stress disorder, the system comprising:
an MRI scanner to obtain first fMRI images of selected regions of the brain of the individual while subjecting an individual to a potential stress causing event; and
a classifier to compare the first fMRI images obtained to second fMRI images obtained from individuals exposed to normal or happy events, to enable a practitioner to determine whether the first fMRI images detect functional connectivity between selected regions of the brain indicative of the individual having PTSD, or acute stress disorder.

16. The system of claim 15, wherein the selected regions of the brain include at least one of the ventral and dorsal visual pathways (VVP & DVP), limbic/paralimbic system (LPS), lateral genituclate nucleus (LGN) and prefrontal cortex (PFC).

17. The system of claim 15, wherein the functional connectivity comprises functional connectivity within at least one of the visual pathways, thalamic, limbic/paralimbic and prefrontal brain networks.

18. The system of claim 15, wherein the magnitude of the change in functional connectivity enables a practitioner to determine whether the individual has PTSD, or acute stress disorder.

19. The system of claim 15, wherein the potential stress causing event is an image of a person under stress.
